(19) United States
(12) Patent Application Publication
(10) Pub. No.: US 2005/0004075 A1
(43) Pub. Date: Jan. 6, 2005

(54) Composition for regulating animal growth, method of manufacture and use thereof

(75) Inventors: Francis Chi, Tsimshatsui (HK); Qin Tang Wen, Tsimshatsui (HK); Tian Shui Lu, Tsimshatsui (HK)

Correspondence Address: WILLIAM J. SAPONE;COLEMAN SUDOL SAPONE P.C., 714 COLORADO AVENUE, BRIDGE PORT, CT, 06605, US

(73) Assignee: WALCOM ANIMAL SCIENCE (I.P.2) LIMITED, Tsimshatsui (HK)

(21) Appl. No.: 10/885,261

(22) Filed: Jul. 6, 2004

Related U.S. Application Data
(63) Continuation-in-part of application No. 10/433,584, filed on Sep. 10, 2003, now abandoned.

(30) Foreign Application Priority Data
Dec. 13, 2000 (CN) CN 00 1 32107.2

(51) Int. Cl. A61K 31/724; A61K 31/13

(52) U.S. Cl. 514/58; 514/665

(57) ABSTRACT

A method of preparing a composition for regulating animal growth comprising the steps of preparing cysteamine or its salt, and mixing the cysteamine or its salt with cyclodextrin or its derivative in a reactor.

### CROSS REFERENCE TO RELATED APPLICATIONS

The present application is a continuation-in-part application of U.S. application Ser. No. 10/433,584, filed Jun. 4, 2003, based on International Application No. PCT/EP01/14628 filed Dec. 12, 2001.

### FIELD OF INVENTION

The present invention relates to a cysteamine-containing composition for regulating growth of animals including but not limited to swine, rabbits, quails, sheep, cattle and chickens. The present invention also relates to a method of preparing the composition, an animal feed additive and an animal feed, and use of such composition for the manufacture of the animal feed additive and animal feed.

### BACKGROUND OF THE INVENTION

It has long been established that growth hormones play an important role in regulating growth of animals. For instance, administering growth hormones in meat producing animals will increase their body weight including their muscle mass. However, there are a number of disadvantages in using growth hormones directly in increasing meat production in these animals. Firstly, growth hormones from different animals are seldom homogenous and different animals (e.g. mammalian animals) only react to certain types of specific growth hormones. Since suitable exogenous growth hormones are normally extracted from pituitary glands, it is rather difficult and uneconomical to prepare sufficient quantity of suitable exogenous growth hormones for use on a large-scale application. Although exogenous growth hormones can now be prepared using DNA recombinant technology, exogenous growth hormones manufactured by such method are still rather expensive. Secondly, the administration of exogenous growth hormones into farm animals is normally performed by direct injection, which is inevitably rather costly and difficult to administer in a large farm. Thirdly, it is rather difficult to control the dose administered to produce precisely the desired effect, and an overdose of exogenous growth hormones is likely to be harmful to the animals. Fourthly, residuals of these exogenous growth hormones may be passed to the meat products and subsequently to humans through consumption thereof. Further studies in this regard are required although some scientists are concerned about the negative side effects of these exogenous growth hormones to humans.

Cysteamine is a component of co-enzyme A and works as a physiological regulator. Cysteamine has been used as an additive in feed in promoting growth of meat producing animals. U.S. Pat. No. 4,711,897 discloses animal feed methods and feed compositions comprising cysteamine. However, it has been identified that cysteamine is a fairly sensitive and unstable compound under normal room temperature conditions. For example, cysteamine is readily oxidized when exposed to air or at an elevated temperature. Cysteamine is highly hydroscopic. Also, cysteamine is unpalatable when taken directly by mouth. Further, ingesting cysteamine directly will cause undesirable gastro side effects. For these reasons, the use of cysteamine had for a long time been limited to direct injection of cysteamine-containing solution into meat producing animals. Effective and large scale application of using cysteamine in promoting growth of farm animals has thus been impractical.

Hence, there continues to exist a need for a composition for regulating and/or promoting growth in animals, and particularly farm animals.

It is thus an object of the present invention in which the above issues are addressed, or at least to provide a useful alternative to the public. Preferably, the composition is safe to administer and easy to formulate with a wide variety of animal feeds.

### SUMMARY OF INVENTION

According to a first aspect of the present invention, there is provided a method of preparing a composition for regulating animal growth (or an animal growth regulating composition) comprising the steps of preparing cysteamine or its salt, and mixing the cysteamine or its salt with cyclodextrin or its derivative in a reactor. One of the main reasons of using cyclodextrin is to reduce the serious toxicity of cysteamine. Preferably, the mixing may be performed under the protection of an inert substance. The composition may preferably be used on multi-gastric animals (e.g. cattles, sheep) because these animals have multiple stomachs and the use of cysteamine alone would be undesirable or may even be unworkable due to the adverse side effects of cysteamine.

The method may comprise heating the cysteamine or its salt and the cyclodextrin or its derivative while mixing for a period of time at a temperature of substantially 25 to 40° C. The method may also comprise stirring the cysteamine or its salt and the cyclodextrin or its derivative to form a first mixture. Preferably, the method may comprise drying the first mixture at a temperature of substantially 40 to 50° C., and this is preferably performed in vacuum. The method may also comprise grounding and/or sieving the first mixture through a mesh screen (e.g. 40-mesh, which means that there are forty pores for each square inch in the mesh screen) to form a second mixture. While a 40-mesh screen may be used, a screen with different mesh size may also be used depending on the size of granules of the composition desired. The second mixture may then be mixed with at least one of fillers, disintegrants and binders to form a third mixture which may be pelleted to form the granules. Thereafter, coating materials made of ingredients selected from a group including cellulose acetate phthalate, polyethylene glycol terephthalate, ehtyl acetate and isopropyl acetate may be applied on the granules.

Preferably, the composition may comprise 1 to 95 wt % of said predetermined amount of cysteamine or its salt. More particularly, the composition may comprise 75 wt % of said predetermined amount of cysteamine or its salt. Advantageously, the composition in the form of granules may have a size ranging from 0.28 to 0.90 mm in diameter.

According to a second aspect of the present invention, there is provided a composition made according to the method as described above. The composition may preferably be administered to the animals by ingestion and in a sufficiently high dose to effect the significant growth promotion.

According to a third aspect of the present invention, there is provided a composition for regulating animal growth (or an animal growth regulating composition) comprising 1 to 95 wt % of cysteamine or its salt and inclusion compound host materials including a stabilizer selected from a group including cyclodextrin or its derivative.

Advantageously, the stabilizer may comprise substantially the cyclodextrin or its derivative.

Preferably, the composition may comprise 1 to 75 wt % of the cysteamine or its salt, and more preferably, the composition may comprise 1 to 40 wt % cysteamine or its salt.

Preferably, the composition may comprise 1 to 60 wt % of the inclusion compound host materials. More preferably, the composition may comprise 10 to 40 wt % of the inclusion compound host materials.

Suitably, the stabilizer of the inclusion compound host materials may be selected from a group including β-cyclodextrin (β-CD), methyl β-cyclodextrin (M-β-CD), hydropropyl β-cyclodextrin (HP-β-CD), hydroethyl β-cyclodextrin (HE-β-CD), poly-cyclodextrin, ehtyl β-cyclodextrin (E-β-CD) and branched cyclodextrin.

Suitably, the composition may comprise at least one of fillers, disintegrants, binders, flavorings, smelling agents and coating materials. The coating materials may represent 1 to 20 wt % of the composition. Preferably, the coating materials may represent 1 to 15 wt % of the composition. The coating materials may be enteric-coated. The coating materials may be selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymer, takh and formogelatine.

Preferably, the composition may comprise the fillers which may be selected from a group including powdered cellulose, starch, and calcium sulfate. In particular, the composition may comprise 1 to 90 wt % of the fillers. More particularly, the composition may comprise 1 to 60 wt % of the fillers.

Preferably, the composition may comprise 5 to 50 wt % of the binders and disintegrants which may be selected from a group including hydropropyl starch, microbial alginate, micro-crystalline cellulose and starch. In particular, the composition may comprise 15 to 35 wt % of the binders and the disintegrants.

Advantageously, the composition may comprise 0.05 to 0.3 wt % of the flavoring and smelling agents for enhancing the flavor of the composition.

Suitably, the composition may be formed into granules, each of which may comprise at least one or more layers of the coating materials. In particular, in each of the granules, the cysteamine or its salt is shielded from its surroundings by the inclusion compound host materials. Each of the granules of the composition may have a size ranging from 0.28 to 0.90 mm in diameter.

Advantageously, each granule of the composition may be encapsulated by the enteric coating materials.

According to a fourth aspect of the present invention, there is provided an animal feed additive comprising a composition as described above.

According to a fifth aspect of the present invention, there is provided an animal feed comprising a composition as described. Suitably, the animal feed may comprise 250 to 700 mg/kg of the composition.

According to a sixth aspect of the present invention, there is provided the use of a composition as described above for the manufacture of an animal feed additive.

According to a seventh aspect of the present invention, there is provided the use of a composition as described above for the manufacture of an animal feed.

According to an eighth aspect of the present invention, there is provided a method of preparing an animal feed comprising a step of mixing a composition as described above with a basal feed (or diet).

### DETAILED DESCRIPTION OF THE PRESENT INVENTION

The present invention is based on the demonstration that a cysteamine-containing composition when ingested by farm animals has activity in increasing body weight thereof. Prior to this finding, there was no suggestion or sufficient indication that administration of the cysteamine-containing composition in animals might have such activity in effectively and safely increasing their body weight in a large-scale application. The present invention also provides a method of manufacture and a use of the cysteamine-containing composition for raising animals by feeding a basal feed (or diet) mixed with the cysteamine-containing composition in order to increase the body weight thereof. The invention may be practised by directly mixing the cysteamine-containing composition with a suitable basal feed. Alternatively, the invention may be practiced by mixing firstly a premix made of the cysteamine-containing composition and other ingredients, and secondly the premix with a suitable basal feed to form a final feed. A basal feed is a diet that an animal is normally fed with. Different animals will require different basal feed. For example, the basal feed for quails normally comprises mainly corn feed.

It is believed that cysteamine having a physiological activity acts as a growth stimulator. Natural cysteamine is a part of coenzyme A (also know as CoA-SH or CoA) which is a coenzyme pattern of pantothenic acid. In the course of metabolism, coenzyme A acts as the carrier of dihydrosulfuryl or variants of hydrosulfuryl which is linked with the hydrosulfuryl of coenzyme A. Experiments performed on animals such as pigs, poultry, fowls, goats, rabbits and fishes have shown that cysteamine can deplete somatostatin (SS) in organisms, and in particular in the median eminence nerve terminal and periventricular nuclear neure soma. This increases the level of growth hormone in the blood of the animals which at the same time raises the level of various other growth stimulating factors including insulin-like growth factor I (IGF-I), insulin, triiodothyronine (T3), trthyroxine (T4) and beta-endorphin (beta-END).

With the increase of these various growth promoting factors, the digestive metabolic rate of the animal is correspondingly increased. It is understood that the general protein synthesis rate of the animal is accordingly increased.

The regulation of the physiology by cysteamine is further explained as follows.
(i) Cysteamine can improve somatostatin metabolism and transportation and promote degradation of somatostatin by effecting the vesicles for storing somatostatin.
(ii) Cysteamine can change the structure and conformation of somatostatin by affecting the dimercapto bonds formed at 3- and 14-positions of SS-14 as well as at 17- and 18-positions of SS-28. This is important in regulating the bioactivity and immuno-reactivity of the physiology of the animal.
(iii)Cysteamine can regulate somatostatin receptors, and reduce the affinity of gastric mucosa cell receptors of the animals fed with the cysteamine-containing composition. Cysteamine is an ingredient for forming coenzyme A which can exhaust somatostatin in tissue organs and in the bloodstream of the animals. Cysteamine can also promote synthesis and release of endogenous growth hormone, regulate production of nerve endocritic hormone, enhance basal level, peak value and total level of various growth hormone.

The novel cysteamine-containing composition prepared according to the present invention comprises two main ingredients of 1 to 95 wt % of cysteamine (or its salts, for example, cysteamine hydrochloride, or other pharmaceutically acceptable acid addition salts thereof) and 1 to 80 wt % of a carrier such as inclusion compound host materials. The chemical formula of cysteamine is HSCH₂CH₂NH₂. The term “cysteamine” referred hereinafter means cysteamine and/or its salt like compound. Cysteamine and its salt are well known in the chemical literature.

The general chemical formula of a cysteamine salt is C₂H₇NS.X, where X may be HCl, H₃PO₄, bitartrate, salicylate, etc. The cysteamine used is preferably of pharmaceutically acceptable standard and the content of carbon, hydrogen, nitrogen and sulfur therein are substantially 31.14 wt %, 9.15 wt %, 18.16 wt % and 41.56 wt % respectively. While the workable content of cysteamine in the cysteamine-containing composition ranges from 1 to 95 wt %, a preferable range of 1 to 75 wt % and a more preferable range of 1 to 40 wt % of cysteamine may be used. Cysteamine is one of the main active ingredients of the cysteamine-containing composition. However, it has been identified that if the content of cysteamine in the cysteamine-containing composition exceeds 95 wt %, mixing the composition with a basal feed would be rather difficult and the effect of the composition for regulating growth of animals would be hindered.

The inclusion compound host materials comprise mainly cyclodextrin and/or its derivative which are selected from a group included methyl β-cycoldextrin (M-β-CD), hydropropyl β-cycoldextrin (HP-β-CD), hydroethyl β-cycoldextrin (HE-β-CD), polycyclodextrin, ethyl β-cyclodextrin (E-β-CD) and branched cycoldextrin. The general chemical formula of cyclodextrin is (C₆O₅H₉)ₙ.(C₆O₅H₉)₂ and the structural formula is as follows. 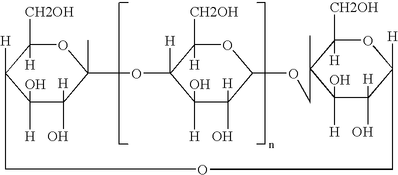
where α-CD n=4; β-CD n=5; γ-CD n=6. (Cyclodextrin is a cyclic oligomer of alpha-D-glucopyranose.)

It is worthwhile to note that the β-CD form of cyclodextrin is preferably used because the internal diameter of its molecule is about 6-8 Å which makes it a particular suitable candidate as an inclusion compound host material for preparation of the cysteamine-containing composition, which involves the use of an inclusion process. The term “cyclodextrin” referred hereinafter means cyclodextrin and/or its derivative. Any derivative of cyclodextrin which has the property of stabilizing and protecting cysteamine from degradation may be used. For example, any one of the group of cyclodextrin or its derivative mentioned above may be used.

While the workable content of the inclusion compound host materials in the cysteamine-containing composition ranges from 1 to 80 wt %, a preferable workable range of 1 to 60 wt % and a more preferable workable range of 10 to 40 wt % of the inclusion compound host materials may be also be used. The actual amount of the inclusion compound host materials used will depend on the actual content of the cysteamine used in preparing the cysteamine-containing composition.

The cysteamine-containing composition may also comprise 1 to 90 wt % of fillers although a preferable workable range of 1 to 60 wt % and a more preferable workable range of 1 to 40 wt % of the fillers may also be used in the composition. The actual content will depend on the actual amount of cysteamine and inclusion compound host materials used. The fillers may be selected from a group including powdered cellulose, starch and calcium sulfate (e.g. CaSO₄.2H₂O). It is to be noted that if the content of the fillers exceeds 90 wt % in the cysteamine-containing composition, the content of the main active ingredients will thus be reduced, and the cysteamine-containing composition may become ineffective in regulating growth of the animals fed with a feed mixed therewith.

The cysteamine-containing composition may also comprise 5 to 50 wt % of disintegrants and binders although a preferable workable range of 10 to 40 wt % and a more preferable workable range of 15 to 35 wt % may also be used. The actual content will depend on the actual amount of cysteamine, the inclusion compound host materials and other ingredients used. The binders and disintegrants may be selected from a group including hydropropyl starch, microbial alginate, microcrystalline cellulose and starch. It has been identified that if the content of the disintegrants and binders in the composition is less than 5 wt %, granules of the composition produced will lack the required hardness. In addition, manufacturing of the composition would become very difficult. If however the content of the disintegrants and binders is more than 50 wt %, the resulting composition will have excessive hardness, this is especially so if the content of binders represent a large portion of the mixture of the disintegrants and binders. This will result in difficult absorption of the composition by the intestines of the animals.

The cysteamine-containing composition may also comprise 0.05 to 0.3 wt % of flavoring and smelling agents which may be a flavoring essence.

The cysteamine-containing composition may also comprise 1 to 20 wt % of coating materials although a preferable workable range is 1 to 15 wt % and a more preferable workable range is 2 to 10 wt %. The actual content will depend on the actual amount of cysteamine, the inclusion compound host materials and the other ingredients used. The coating materials are preferably enteric-coated which allows dissolution in an alkaline environment such as in the intestines. The coating materials may be selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymers, takh and formogelatine. It has been identified if the content of the coating materials is less than 1 wt %, granules of the composition may not be entirely covered by the coating materials which act as a protective layer. The cysteamine-containing composition may thus degrade before being absorbed by the intestines into the bloodstream of the animals. On the other hand, if the content of the coating materials exceeds 15 wt %, the active ingredients in the composition may not effectively be released from the composition. Thus, the intended regulation of growth would be not achieved. In any event, it has been identified that an animal feed comprising 250 to 700 mg/kg of the composition is effective, when fed to the animal, in increasing its body weight.

The cysteamine-containing composition made according to the present invention is in the form of small granules each of which has a preferable diameter of substantially 0.28 to 0.90 mm. These granules are prepared using a micro-encapsulation method. The method involves using a macromolecular substance having inclusion property. One substance which may be used is the inclusion compound host materials (which comprises mainly cyclodextrin) described above. The inclusion compound host materials are a macromolecular substance which acts as a molecular capsule to engulf the molecules of cysteamine, whereby cysteamine in the composition is protected and insulated from light, heat, air and moisture of the surroundings. The stability of cysteamine is thus preserved. The inclusion compound host materials used in the micro-encapsulation method is preferably a cyclic polysaccharide compound having 6 to 12 glucose molecules, which is produced by reacting cyclodextrin glycosidtransferase and starch in the presence of Bacillus. Various studies using acute, subacute and chronic toxic tests have shown that the macromolecular substance is non-toxic. Subsequent to the micro-encapsulation process, each granule may be coated with at least one and preferably a plurality of layers of the coating materials described above. The following provides a more detailed description of one embodiment of a method of preparing the cysteamine-containing composition according to the present invention.

In a jacketed reactor linked with polytetrafluoroethylene and equipped with a polytetrafluoroethylene coated stirrer, 4080 g of 75 wt % cysteamine hydrochloride solution in ethanol is added with mainly nitrogen being the atmosphere. The purity, melting point and burning residue of the cysteamine used are preferably 98% or above, 66 to 70° C. and 0.05% or below respectively. 1200 g β-cyclodextrin is then added into the reactor similarly under the protection of nitrogen gas. (The quality of β-cyclodextrin is in accordance with the requirements for a food additive. In particular, the dry basis purity is more than 98%; the weight loss by drying is less than 10.0%; the burning residue is less than 0.2%; the content of heavy metal is less than 10 ppm; the arsenic content is less than 2 ppm.) The mixture is then heated for 3 hours at 40° C. Heating is then stopped and stirring continues for two hours thereafter, products resulted therefrom are then grounded and sieved through a screen (e.g. 40-mesh) filter after the products have been vacuum dried at a temperature of 40-50° C. All parts of the equipment, which may come in contact with the ingredients of the composition, should preferably be made of stainless steel.

In a tank-type mixer, 4200 g (on dry basis) of the cysteamine which has undergone the inclusion process as described, 2600 g of the fillers, and 1200 g of the disintegrants and 1700 g binders are added under the protection of a dry surroundings. These ingredients are then thoroughly mixed, and a suitable amount of anhydrous ethanol may be added and then mixed therewith. The resulting mixture presents a soft material with moderate hardness, so that it can be shaped into a ball by a light hold of palms. The ball-shaped resulting mixture may then be broken up by a light touch. After the mixture is pelleted by a granulator under the protection of nitrogen, the small granules resulting therefrom is immediately introduced to a fluid-bed dryer, and is then dried at the temperature of 40-50° C. in a substantially vacuum environment.

Enteric coating materials are then prepared by a method with the following formulation: cellulose acetate phthalate 8.0 g, polyethylene glycol terephthalate 2.4 ml, ethyl acetate 33.0 ml and isopropyl acetate 33.6 ml. The resultant granules obtained above are uniformly coated under the protection of nitrogen with at least one layer but preferably a plurality of layers the enteric coating materials described above. The enteric coating materials are dissolvable only at an alkaline environment. This can prevent the cysteamine from prematurely escaped from the composition while it is still in the stomach of the animal. Cysteamine can adversely stimulate gastric mucous of the stomach of the animals.

The resultant granules of the cysteamine-containing composition are then dried completely in a substantially vacuum dryer at a temperature of 40 to 50° C. Then, all solvents are removed. The resultant granules are then allowed to cool to room temperature, the micro-capsula were mixed with a suitable amount of flavoring and smelling agents by a cantilever double helix blender. The cystreamine-containing composition is a microcapsule with its interior having cysteamine hydrochloride and cyclodextrin, and with its exterior coated with the enteric coating materials.

The composition produced will exhibit small granular (or micro-particulate) shape having smooth surface, good flow property, and is easy to be blended with various animal feeds. The diameter of each granule of the composition is preferably 0.28 to 0.90 mm. The composition also has excellent stability. It has been found that after the composition is packaged with sealed plastic bags and stored for one year in a cool, dark and dry place, their properties remain unchanged. Therefore, they meet the requirements for a feed additive.

The composition having the particular construction described above has a number of functional advantages over cysteamine by itself. Firstly, the activity of the cysteamine contained in the composition is preserved after it has been produced. This is important as feed additive such as the composition may be stored for a relatively long period of time before use. Secondly, the composition does not cause any noticeable gastro side effects to the animals fed therewith. Thirdly, the activity of the composition is preserved not only during storage but more importantly until it reaches the intestines of the animals. Fourthly, the composition can be easily administered to farm animals on a large scale basis cost-effectively because it can be readily mixed with any basal feed. No separate procedure or injection is needed at all. A number of experiments had been performed to demonstrate the effect of administering the cysteamine-containing composition to the diet of various farm animals.

### EXPERIMENT 1

The cysteamine-containing composition used in this experiment included 30 wt % cysteamine, 20 wt % of the inclusion host compound materials and the coating materials, 26 wt % of the fillers, 23.9 wt % of the disintegrants and binders and 0.1 wt % of the flavoring and smelling agents. It is to be noted that the composition in the experiment comprises 12 to 17 wt % of the inclusion host compound materials including mainly cyclodextrin and 1 to 5 wt % of the coating materials.

The test animals were weaning piglets of about 35 days old. There was a test group of 80 weaning piglets and a control group of 80 weaning piglets. The test piglets were fed with a basal feed added with 500 mg/kg of the cysteamine-containing composition. The piglets in the control group were fed with the same basal feed but without the cysteamine-containing composition. The duration of the experiment was 28 days.

Results:

It was recorded that the mean daily gain in body weight of each piglet in the test group was 512 g while that of in the control group was 456 g. It is calculated that the mean daily gain in body weight of each piglet in the test group is 12.28% more than that in the control group.

### EXPERIMENT 2

The same cysteamine-containing composition used in Experiment 1 was used in this experiment.

The test animals were growing pigs of about 50 to 90 days old. There was a test group of 100 growing pigs and a control group of 100 growing pigs. The test pigs were fed with a basal feed added with 700 mg/kg of the cysteamine-containing composition. The pigs in the control group were fed with the same basal feed without the cysteamine-containing composition. The duration of the experiment was 95 days.

Results:

It was recorded that the average daily increase in body weight of each pig in the test group was 842 g while that of in the control group was 747 g. It is calculated that the increase in body weight of each pig in the test group is 12.72% more than that in the control group.

### EXPERIMENT 3

The same cysteamine-containing composition used in Experiment 1 was used in this experiment.

The test animals were New Zealand rabbits. There was a test group of 29 rabbits and a control group of 14 rabbits. All the rabbits were 55 days old. The test rabbits were fed with a basal feed added with 300 mg/kg of the cysteamine-containing composition. The rabbits in the control group were fed with the same basal feed without the cysteamine-containing composition. All other conditions for the two groups of rabbits were identical. The duration of the experiment was 66 days.

Results:

It was recorded that the mean daily gain in body weight of each rabbit in the test group during the experiment period was 1061.8 g while that of in the control group was 840.1 g. The mean daily gain in body weight of each rabbit in the test group was 26.4% more than that in the control group.

### EXPERIMENT 4

The same cysteamine-containing composition used in Experiment 1 was used in this experiment.

The test animals in this experiment were lambs (including New Zealand merino (♂)× local native sheep (♀) of second filial generation (F₂)). There was a test group of 14 lambs and a control group of 14 lambs. All of the lambs were three months old at the beginning of the experiment. The test lambs were fed with a basal feed added with 250 mg/kg of the cysteamine-containing composition. The lambs in the control group were fed with the same basal feed but without the cysteamine-containing composition. All other conditions for the two groups of lambs were identical. The duration of the experiment was 56 days.

Results:

It was recorded that during the experiment the mean daily gain in body weight of each lamb in the test group was 70.8 kg. The mean daily gain in body weight of each lamb in the test group was calculated to be 15.08% more than that in the control group.

### EXPERIMENT 5

The same cysteamine-containing composition used in Experiment 1 was used in this experiment.

The test animals were weaning piglets which were the offspring of Large White and Landrace. The experiment was performed in the Esconde Farm in the Philippines during the 28-day period from 24 April to 21 May 2000. There was a test group of 80 weaning piglets and a control group of 80 weaning piglets. The weaning piglets were all 35 days old and each of the piglets was about 8.6 kg in initial weight. The test weaning piglets were fed with a basal feed added with 500 ppm the cysteamine-containing composition. The weaning piglets in the control group were fed with the same basal feed but without cysteamine-containing composition. All other conditions for the two groups of weaned piglet were identical. The following table shows the nutritional content of the daily basal feed.

TABLE 1
Content of basal feed

| | | |
|---|---|---|
| Total energy M.E. | 3250 | kcal/kg |
| (metabolic energy) | | |
| Crude protein | 20 | wt % |
| Calcium | 0.95 | wt % |
| Phosphorous | 0.54 | wt % |
| Crude fiber | <3 | wt % |
| Amino acid: lysine | 1.40 | wt % |
| Amino acid: | 0.77 | wt % |
| methionine & | | |
| cysteine | | |

Results:

At the end of the experiment, it was recorded that the average body weight of the piglets in the control group was 21.4 kg which translates to 456 g in daily weight gain. The average body weight of the piglets in the test group was 23 kg which translates to 512 g in daily weight gain. It is calculated that the piglets in the test group has 12.28% more weight gain than those in the control group.

The feed conversion efficiency of the piglets in the test group was 1.37 and that in the control group was 1.41. The feed conversion efficiency of 1.37 means that for every weight gain of 1 kg, 1.37 of feed is consumed. In other words, the piglets in the test group have a 2.13% higher feed conversion efficiency than those in the control group.

It is to be noted that the piglets in the test group had reduced consumption in feed by 432 g. There was therefore a total reduction of 34.56 kg in feed consumption by the 80 test piglets.

### EXPERIMENT 6

The same cysteamine-containing composition used in Experiment 1 was used in this experiment.

The test animals were fattening pigs of about 35 to 90 days old, which were the offspring of Large White and Landrace. The experiment was performed in the Rocky Farm in the Philippines during a 95-day period. There were a test group of 100 pigs and a control group of 100 pigs. The pigs in the test group were 23.3 kg on average and those in the control group were 23.6 kg at the beginning of the experiment. The pigs in the test group were fed with a basal feed added with 700 ppm the cysteamine-containing composition. The pigs in the control group were fed with the same basal feed except without cysteamine-containing composition. All other conditions for the two groups of pigs were identical. The following table shows the content of the daily basal feed.

TABLE 2
Content of basal feed

| | Grower Diet | Finisher Diet |
|---|---|---|
| Energy M.E. | 3150 kcal/kg | 3100 kcal/kg |
| Crude protein | 18% | 16% |
| Calcium | 0.85% | 0.75% |
| Phosphorous | 0.52% | 0.50% |
| Crude protein | <4.0 | <5.0 |
| Amino acid: lysine | 1.2% | 1.09% |
| Amino acid: | 0.65% | 0.60% |
| methionine & | | |
| cysteine | | |

Results:

At the end of the experiment, it was recorded that the average body weight of the pigs in the control group was 94.6 kg which translates to 747 g in average daily weight gain. The average body weight of the pigs in the test group was 103.9 kg which translates to 842 g in daily weight gain. It is calculated that the piglets in the test group has 12.72% more weight gain than those in the control group. The feed conversion efficiency of the pigs in the test group was 2.36 and that in the control group was 2.66. In other words, the pigs in the test group have a 1.13% higher feed conversion efficiency than those in the control group.

It is to be noted that each pig in the test group had reduced consumption in feed by 24.81 kg . There was therefore a total reduction of 2,418 kg feed consumption by the 100 test pigs.

### EXPERIMENT 7

The same cysteamine-containing composition used in Experiment 1 was used in this experiment.

The experiment was performed at the Laboratory of Physiology and Biochemistry in the Nanjing Agricultural University from 5 December to 28 December 2000. The test animals were quails. 160 fifteen-day old quails were used. The experiment began when the quails reached seventeen days old and ended when they reached thirty-eight days old.

The quails were randomly divided into eight groups. Three types of diet were prepared with different amount of the cysteamine-containing composition. The diet included mainly basic corn feed. All groups of quails were allowed unrestricted access and amount of their respective diet. Table 3 below summarizes the diet and the average weight of the quails before and after the experiment.

TABLE 3
Effect of the cysteamine-containing composition on
growth of quails.

| | | Basal feed | | |
|---|---|---|---|---|
| | | with varying | | |
| | | amount of | | |
| | Age of | the | | |
| | quails when | cysteamine- | Average | Average |
| | fed with the | containing | weight of | weight of |
| | cysteamine- | composition | quails | quails |
| | containing | (basal feed: | before | after |
| Group | diet | corn) | experiment, g | experiment, g |
| 1 | — | 0 ppm | 60.34 ± 7.56 | 177.35 ± 18.32 |
| 2 | — | 0 ppm | 59.86 ± 8.01 | 173.53 ± 12.76 |
| 3 | 17-38 | 200 ppm | 59.22 ± 8.38 | *153.42 ± 14.94 |
| 4 | 17-38 | 300 ppm | 58.03 ± 7.26 | 179.45 ± 15.45 |
| 5 | 24-38 | 200 ppm | 59.99 ± 8.35 | *194.08 ± 17.93 |
| 6 | 24-38 | 300 ppm | 58.72 ± 9.65 | *210.06 ± 24.71 |
| 7 | 31-38 | 200 ppm | 59.26 ± 7.78 | 167.64 ± 18.55 |
| 8 | 31-38 | 300 ppm | 60.86 ± 9.05 | 168.57 ± 17.97 |

Groups 1 and 2 were the control groups. Their diet included the basal feed including mostly corn and contained no cysteamine-containing composition. Groups 3 to 8 were the test groups. Their diet included the same basal feed but added with different amount of the cysteamine-containing composition as indicated in column 3.
Results and Discussion:

Comparing the average body weight of the quails in Groups 5 and 6 with Groups 1 and 2, it is shown that quails fed with the cysteamine-containing composition through their diet starting at the age of 24 days old does increase their body weight by 10.65% and 19.7%. More particularly, the quails in Group 6 had a higher increase in body weight than those in Group 5. This can be explained by the fact that the diet of the quails in Group 6 had a higher content of the cysteamine-containing composition.

Comparing the average body weight of the quails in Groups 7 and 8 with those in Groups 1 and 2, it is shown that the presence of the cysteamine-containing composition does not assist in increasing the body weight of the quails in Groups 7 and 8. Comparing the average body weight of the quails in Groups 3 and 4 with those in Groups 1 and 2, it is shown that the presence of the cysteamine-containing composition did not assist in increasing the body weight of the quails in Groups 3 and 4.

The effect of the cysteamine-containing composition on the quails from different groups is explained as follows. The actively growing stage of quails is a fairly short. The cysteamine-containing composition is effective in increasing the body weight while the animal is in its growing stage. Administration of the cysteamine-containing composition beginning at a very early stage of its development (e.g. before 17 days of age) does not assist in increasing its body weight. For this reason, the quails in Groups 1 & 2 and 3 & 4 were similar in their body weight after the experiment. Similarly, administration of the cysteamine-containing composition beginning at a very late stage of its development (e.g. after 31 days of age) does not assist in increasing its body weight. The administration of the cysteamine-containing compound should preferably be initiated at the mid- to final stage of the growth of the animal. In Groups 3 and 4, the administration of the cysteamine-containing composition initiated at a development stage which was too early. This might have lowered the level of growth receptors and it may have caused the cysteamine-containing composition to appear to have no impact in increasing the body weight of the animals.

The contents of each of the references discussed above, including the priority application and the references cited therein, are herein incorporated by reference in their entirety. It is to be noted that numerous variations, modifications, and further embodiments are possible and accordingly, all such variations, modifications and embodiments are to be regarded as being within the scope of the present invention and to be understood by the persons skilled in the art.

1. A use of a composition for regulating growth in animals comprising a step of producing said composition including 1 to 95 wt % of cysteamine or its salt and an inclusion compound host materials composition including a stabilizer selected from a group including cyclodextrin or its derivative and a further step of orally administering said composition to said animals.

2. The use according to claim 1, wherein said animals are multi-gastric animals.

3. The use according to claim 2, wherein said animals are cattle.

4. The use according to claim 1, wherein said animals are swine, rabbits, quails, sheep or chickens.

5. The use according to claim 1, wherein said composition is an animal growth regulating composition comprising 1 to 75 wt % of cysteamine or its salt.

6. The use according to claim 5, wherein said animal growth regulating composition comprises 1 to 40 wt % cysteamine or its salt.

7. The use according to claim 1, wherein said animal growth regulating composition comprises 1 to 60 wt % of said inclusion compound host materials composition.

8. The use according to claim 7, wherein said animal growth regulating composition comprises 10 to 40 wt % of said inclusion compound host materials composition.

9. The use according to claim 1, wherein said stabilizer is selected from a group including cyclodextrin (-CD), methyl β-cyclodextrin (M-β-CD), hydropropyl β-cyclodextrin (HP-β-CD), hydroethyl β-cyclodextrin (HE-β-CD), poly-cyclodextrin, ehtyl β-cyclodextrin (E-β-CD) and branched cyclodextrin.

10. The use according to claim 1, comprising a step of introducing at least one of fillers, disintegrants, binders, flavorings and smelling agents, and coating materials for producing said animal growth regulating composition.

11. The use according to claim 1, wherein said animal growth regulating composition comprises coating materials.

12. The use according to claim 10 or 11, wherein said animal growth regulating composition comprises 1 to 20 wt % of said coating materials.

13. The use according to claim 10 or 11, wherein said coating materials are enteric.

14. The use according to claim 10 or 11, wherein said coating materials are selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymer and formogelatine.

15. The use according to claim 10, wherein said animal growth regulating composition comprises 1 to 90 wt % of said fillers.

16. The use according to claim 15, wherein said animal growth regulating composition comprises 1 to 60 wt % of said fillers.

17. The use according to claim 10, wherein said fillers are selected from a group including powdered cellulose, starch, and calcium sulfate.

18. The use according to claim 10, wherein said animal growth regulating composition comprises 5 to 50 wt % of said binders and said disintegrants.

19. The use according to claim 18, wherein said animal growth regulating composition comprises 15 to 35 wt % of said binders and said disintegrants.

20. The use according to claim 10, wherein said binders and said disintegrants are selected from a group including hydropropyl starch, microbial alginate, microcrystalline cellulose and starch.

21. The use according to claim 10, comprising 0.05 to 0.3 wt % of said flavoring and smelling agents for enhancing the flavor of said animal growth regulating composition.

22. The use according to claim 10 or 11, comprising a step of forming said animal growth regulating composition into granules each of which comprising at least one layer of said coating materials.

23. The use according to claim 1, comprising a step of forming said animal growth regulating composition into granules for shielding the cysteamine or its salt from its surroundings by said inclusion compound host materials composition.

24. The use according to claim 23, wherein each of said granules of said animal growth regulating composition has a size ranging from 0.28 to 0.90 mm in diameter.

25. The use according to claim 13, comprising a step of encapsulating said animal growth regulating composition with said enteric coating materials.

26. The use according to claim 1 for the manufacture of an animal feed.

27. The use according to claim 27, wherein said animal feed comprises 250 to 700 mg/kg of said animal growth regulating composition.

28. An animal growth regulating composition, comprising 1 to 95 wt % of cysteamine or its salt, and an inclusion compound host materials composition comprising a stabilizer selected from a group including cyclodextrin or its derivative.

29. The animal growth regulating composition according to claim 29, wherein said animals are multi-gastric animals.

30. The animal growth regulating composition according to claim 30, wherein said animals are cattle.

31. The animal growth regulating composition according to claim 29, wherein said animals are swine, rabbits, quails, sheep or chickens.

33. The animal growth regulating composition according to claim 29, comprising 1 to 75 wt % of cysteamine or its salt.

34. The animal growth regulating composition according to claim 33, comprising 1 to 40% cysteamine or its salt.

35. The animal growth regulating composition according to claim 29, comprising 1 to 60 wt % of said inclusion compound host materials composition.

36. The animal growth regulating composition according to claim 35, comprising 10 to 40 wt % of said inclusion compound host materials composition.

37. The animal growth regulating composition according to claim 29, wherein said stabilizer is selected from a group including cyclodextrin (-CD), methyl β-cyclodextrin (M-β-CD), hydropropyl β-cyclodextrin (HP-β-CD), hydroethyl β-cyclodextrin (HE-β-CD), poly-cyclodextrin, ehtyl β-cyclodextrin (E-β-CD) and branched cyclodextrin.

38. The animal growth regulating composition according to claim 29, wherein said composition comprises at least one of fillers, disintegrants, binders, flavorings and smelling agents and coating materials.

39. The animal growth regulating composition according to claim 29, wherein said composition comprises coating materials.

40. The animal growth regulating composition according to claim 38 or 39, comprising 1 to 20 wt % of said coating materials.

41. The animal growth regulating composition according to claim 38 or 39, wherein said coating materials are enteric.

42. The animal growth regulating composition according to claim 38 or 39, wherein said coating materials are selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymer and formogelatine.

43. The animal growth regulating composition according to claim 38, comprising 1 to 90 wt % of said fillers.

44. The animal growth regulating composition according to claim 43, comprising 1 to 60 wt % of said fillers.

45. The animal growth regulating composition according to claim 38, wherein said fillers are selected from a group including powdered cellulose, starch, and calcium sulfate.

46. The animal growth regulating composition according to claim 38, comprising 5 to 50 wt % of said binders and said disintegrants.

47. The animal growth regulating composition according to claim 46, comprising 15 to 35 wt % of said binders and said disintegrants.

48. The animal growth regulating composition according to claim 38, wherein said binders and said disintegrants are selected from a group including hydropropyl starch, microbial alginate, microcrystalline cellulose and starch.

49. The animal growth regulating composition according to claim 38, comprising 0.05 to 0.3 wt % of said flavoring and smelling agents for enhancing the flavor of said animal growth regulating composition.

50. The animal growth regulating composition according to claim 38, wherein said composition is formed into granules, each of which comprises at least one layer of said coating materials.

51. The animal growth regulating composition according to claim 50, being formed into granules in which the cysteamine or its salt is shielded from its surroundings by said inclusion compound host materials composition.

52. The animal growth regulating composition according to claim 50, wherein each of said granules has a size ranging from 0.28 to 0.90 mm in diameter.

53. The animal growth regulating composition according to claim 41, wherein said composition is encapsulated by said enteric coating materials.

54. The animal growth regulating composition according to claim 29 for the manufacture of an animal feed additive.

55. The animal growth regulating composition according to claim 29 for the manufacture of an animal feed.

56. The animal growth regulating composition according to claim 55, wherein said animal feed comprises 250 to 700 mg/kg of said regulating composition.

57. The animal feed additive, comprising an animal growth regulating composition as claimed in any one of claims 29 to 56.

58. The animal feed, comprising an animal growth regulating composition as claimed in any one of claims 29 to 56.

59. The animal feed according to claim 58, comprising 250 to 700 mg/kg of said animal growth regulating composition.

60. The animal feed according to claim 58 or 59 further comprising a basal feed.

61. A method of preparing an animal growth regulating composition, comprising the steps of preparing cysteamine or its salt, and mixing the cysteamine or its salt with cyclodextrin or its derivative in a reactor.

62. The method according to claim 61, wherein said animals are multi-gastric animals.

63. The method according to claim 62, wherein said animals are cattle.

64. The method according to claim 61, wherein said animals are swine, rabbits, quails, sheep or chickens.

65. The method according to claim 61, 62 or 63, wherein the mixing of the cysteamine or its salt with the cyclodextrin or its derivative is performed under the proctection of an inert substance.

66. The method according to claim 61, 62 or 63, comprising heating the cysteamine or its salt and the cyclodextrin or its derivative while mixing for a period of time at a temperature of substantially 25 to 40° C.

67. The method according to claim 61, comprising stirring the cysteamine or its salt and the cyclodextrin or its derivative to form a first mixture.

68. The method according to claim 67, sieving said first mixture through a mesh screen to form a second mixture.

69. The method according to claim 68, comprising a step of drying said second mixture at a temperature of 40 to 50° C.

70. The method according to claim 69, comprising s step of mixing said second dried mixture with at least one of fillers, disintegrants and binders to form a third mixture.

71. The method according to claim 70, comprising a step of pelleting said third mixture to form granules.

72. The method according to claim 71, comprising applying on said granules coating materials made of ingredients selected from a group including cellulose acetate phthalate, polyethylene glycol terephthalate, ehtyl acetate and isopropyl acetate.

73. The method according to claim 61, wherein said composition comprises 1 to 95 wt % of the cysteamine or its salt.

74. The method according to claim 62, wherein said composition in the form of granules has a size ranging from 0.28 to 0.90 mm in diameter.
